(19) United States
(12) Patent Application Publication
(10) Pub. No.: US 2011/0229534 A1
(43) Pub. Date: Sep. 22, 2011

(54) Non-Irritating Oral Care Formulations

(75) Inventor: Shanta M. Modak, River Edge, NJ (US)

(73) Assignee: The Trustees of Columbia University In The City Of New York

(21) Appl. No.: 12/444,089

(22) PCT Filed: Oct. 3, 2007
(86) PCT No.: PCT/US07/80274

§ 371 Date: Dec. 1, 2009

Related U.S. Application Data
(60) Provisional application No. 60/850,176, filed on Oct. 5, 2006.

(51) Int. Cl. A61K 31/60; A61K 31/407; A61K 9/68; A61K 9/00; A61P 1/02; A61P 29/00

(52) U.S. Cl. 424/401; 424/55; 424/54; 424/53; 424/48

(57) ABSTRACT

The present invention provides a unique oral care formulation comprising an anti-irritant amount of a salicylate salt and an effective amount of a taste enhancing agent. Certain embodiments further comprise an anti-inflammatory amount of an irritating agent, for example ketorolac. The oral care formulations may be used on various devices to treat and/or prevent mucosal irritations such as ulcerations.

### CROSS-REFERENCE TO RELATED APPLICATIONS

This application claims priority from U.S. Provisional Application Ser. No. 60/850,176, filed Oct. 5, 2006, which is incorporated by reference in its entirety herein.

### FIELD OF THE INVENTION

The present invention relates to oral care compositions and methods for inhibiting oral mucosal irritations. Specifically, the present invention relates to the use of metal salts of salicylic acid to inhibit irritation otherwise caused by oral care agents such as ketorolac.

### BACKGROUND OF THE INVENTION

Periodontal disease includes those diseases which attack the gingiva and the underlying alveolar bone supporting the teeth. Periodontal disease includes a series of diseases exhibiting various syndromes which vary from each other according to the stage or situation of the disease or the age of the patient, and have not been definitely subclassified. The term is used for any inflammatory disease which initially occurs at a marginal gingiva area and may affect the alveolar bone. Two common periodontal diseases are gingivitis (inflammation of the gingiva) and periodontitis (manifested by progressive resorption of alveolar bone, increasing mobility of the teeth, and loss of the teeth at advanced stage). Other terms used for various aspects of periodontal disease include “juvenile periodontitis”, “acute necrotizing ulcerative gingivitis”, and “alveolar pyorrhea”. Periodontal disease is characterized by one or more of the following: inflammation of the gingiva, formation of periodontal pockets, bleeding and/or pus discharge from the periodontal pockets, resorption of alveolar bone, loose teeth and loss of teeth.

Periodontal disease is generally considered to be caused by or associated with bacteria which are generally present in dental plaque which forms on the surface of the teeth and in the periodontal pocket. Known methods for treating periodontal disease often include the use of antimicrobials.

Certain nonsteroidal anti-inflammatory drugs (NSAIDs) have been shown to be useful in the treatment of periodontal disease. Ketorolac, and its pharmaceutically-acceptable, non-toxic esters and salts are well known NSAIDs. Ketorolac tromethamine is a potent non-narcotic analgesic compound with cyclooxiginase inhibitory activity that has been developed for oral and parenteral use. Ketorolac and its esters and salts, particularly the tromethamine salt, are disclosed in the following references: U.S. Pat. No. 4,089,969; Rooks et al., Drugs Experimental Clinical Research, Vol. XI, No. 8 (1985), pp. 479-492; and Mroszcsak, et al., Drug Metabolism and Disposition, Vol. 15, No. 5 (1987), pp. 618-626.

U.S. Pat. No. 5,785,951 discloses methods and compositions for the prevention or treatment of bone loss due to periodontal disease, oral surgery, periodontal surgery, tooth extraction, dental implants or scaling comprising topical administration to mucosal tissues of the oral cavity of a composition having about 0.001% to 0.2% of ketorolac in the oral cavity. The '951 patent teaches that the treatment of periodontal disease by the topical application of ketorolac has been found to not only reduce inflammation caused by the disease but also reduces the rate of alveolar bone resorption characteristic of periodontis. Various oral formulations containing ketorolac (consisting of substantially of the (−) of S enantiomer) have been described in the '951 patent.

Several NSAIDs (e.g., ibuprofen and naproxen) are known to have more than one enantiomeric form which differ in properties from one another. Advantages of certain enantiomeric forms of NSAIDs are disclosed in the following references: PCT Patent Application No. WO/00421; Caldwell et al., Biochemical Pharmacology, Vol. 37 (1988), pp. 105-114; and Hutt et al., Journal of Pharmacy and Pharmacology, Vol. 35 (1983), pp. 693-704.

U.S. patent application publication number 2005/0256197 discloses that chronic periodontitis is also a significant contributor to circulating tumor necrosis factor (TNF). Ketorolac has also been shown to reduce GCF-IL-1 beta in the oral cavity when used as an oral rinse. Accordingly, ketorolac oral rinse (KOR) may also be useful in the management of diabetes patients. However, oral administration of ketorolac was shown to cause gastric mucosal ulceration (J. clinical Pharmacology 1996, 96, 521-539). Furthermore, ulceration in the oral mucosa was observed in a small number of patients who used oral rinse containing ketorolac.

Salicylic acid and acetyl salicylic acid have also been known to have ulcerative properties. Metal salts of salicylic acid such as zinc and copper salicylate are known to have anti-inflammatory and anti-oxidative properties and have been used in oral compositions. Zinc salicylate and other zinc salts (e.g., zinc gluconate and zinc lactate) along with an inorganic fluoride salt has been used in oral compositions for inhibiting odor causing bacteria and preventing plaque formation (see U.S. Pat. No. 4,469,674).

Copper salicylate has been used topically to treat osteoarthritis and other inflammatory diseases. (Sorenson, Prog. in Med. Chem. 1978, 15:211). It has been reported that copper complexes exhibit anti-ulcer activity and are also known to have anti-inflammatory and analgesic activity (Williams et al., J. pharmacological Sci, 1976, 65:126). Copper salicylate containing oral products have been used to prevent plaque formation (U.S. Pat. Nos. 5,628,986 and 4,332,791). West et al. showed that oral administration of copper salicylate is more effective than cimetidine in preventing ulcers induced by the anti-inflammatory drugs aspirin and indomethacin. (West G. B., Methods 1982, 8:33). Anti-inflammatory effects of copper complexes have been well documented. (Chohan et. al., J. of Enzyme inhibition and Medical chemistry 2002, 17:87-91). However, copper salicylate or zinc salicylate have not been evaluated in an oral composition for the prevention of ulcer induced by NSAID drugs such as aspirin or ketorolac.

Thus, there remains a need in the art for oral compositions that prevent or treat mucosal irritation or ulceration. The present invention meets this need with the development of oral formulations containing certain non steroidal anti-inflammatory, anti-irritant compounds such as copper salicylate, zinc salicylate and other zinc salts for reducing the ulcerative effect of ketorolac.

### SUMMARY OF THE INVENTION

The present invention relates to oral care formulations comprising an irritation-inhibiting amount of copper salicylate and/or zinc salicylate. In non-limiting embodiments of the invention, the formulations further comprise an agent which would otherwise cause irritation (an “irritating agent”), such as, but not limited to, ketorolac, peroxide, surfactant, alcohol, microabrasives, or an essential oil such as spearmint, wintergreen, peppermint, or lavender oil.

In certain non-limiting embodiments, the present invention provides for oral care formulations comprising an irritation-inhibiting amount of copper salicylate and/or zinc salicylate as well as an anti-inflammatory amount of ketorolac. Such formulations may be used to inhibit periodontal disease, where the ketorolac-associated side effect of mucosal ulcers is inhibited by the salicylate compound. These formulations may be used to particular advantage by diabetic patients, for whom the use of oral rinses containing ketorolac have inhibited periodontitis but decreased insulin resistance.

In other non-limiting embodiments, the present invention provides for oral care formulations comprising an irritation-inhibiting amount of copper salicylate and/or zinc salicylate which may be used post-operatively in orthodontic surgery patients to reduce inflammation and to reduce the risk of infection.

In various non-limiting embodiments, the present invention provides for oral care formulations for use as oral rinses (mouthwashes), toothpastes, dental adhesives, chewing gum, lozenges, solutions to be applied to the oral mucosa or tongue, or dental powders. The present invention further provides for formulations applied to devices such as dental floss, toothpicks, dentures, braces (retainers), pacifiers or pet toys.

### DETAILED DESCRIPTION

The present invention describes the development of unique oral formulations containing non-steroidal anti-inflammatory, anti-ulcer, anti-irritant compounds such as copper salicylate and zinc salicylate for reducing the ulcerative effect of mucosal irritating substances such as ketorolac. The present invention has discovered unique oral compositions with compounds that exhibit antibacterial and anti-inflammatory activity and therefore can potentiate the efficacy of mucosal irritating substances in preventing or treating oral diseases.

### Compositions

The present invention is directed to the new discovery of the development of oral formulations containing certain non-steroidal anti-inflammatory, anti-irritant compounds, such as zinc salicylate and other zinc salts and/or curcurmin (preferably, purified curcurmin) for reducing the ulcerative effect of mucosal irritating agents such as ketorolac.

In accordance with the present invention, an oral care formulation is provided having an irritation-inhibiting or anti-irritant amount of a salt of salicylic acid and a taste enhancing agent. Examples of such salicylic acid salts include, but are not limited to copper salicylate and/or zinc salicylate. Other zinc salts may also be used in the present invention, including for example zinc lactate or zinc gluconate or an insoluble zinc salt, which have anti-irritant and wound healing properties. Suitable zinc salts for use in these compositions include zinc acetate (molar solubility in water of 1.64 moles/l), zinc butyrate (molar solubility in water of 0.4 moles/l), zinc citrate (molar solubility in water of <0.1 moles/l), zinc gluconate (molar solubility in water of 0.28 moles/l), zinc glycerate (moderately water soluble), zinc glycolate (moderately water soluble), zinc formate (molar solubility in water of 0.33 moles/l), zinc lactate (molar solubility in water of 0.17 moles/l), zinc picolinate (moderately water soluble), zinc proprionate (molar solubility in water of 1.51 moles/l), zinc salicylate (low water solubility), zinc tartrate (moderately water soluble) and zinc undecylenate (moderately water soluble).

The zinc salt may be used in the form of a zinc-hydrogel (zinc gluconate and one or more other zinc salts, panthenol and a hydrogel), which has been shown to reduce skin and mucous membrane irritation resulting from contact with latex materials, chemicals such as nonoxynol-9, detergents used in vaginal lubricants, soaps oral products, and other detergents. (See U.S. Pat. Nos. 5,985,918, 5,965,619; U.S. Patent application publication Nos. 20050048139, and 20040102429; see also Dermatitis 2006, 16:22-27; all incorporated by reference in their entirety). The “irritation-inhibiting” or “anti-inhibiting” amount of the salt is one in which the prevents mucosal irritations including for example ulcerations. In a specific embodiment, the amount of the salicylate salt is between about 0.05% and about 0.3% (w/w). Other zinc salts, including for example zinc hydrochloride may be present in amounts ranging from about 0.1% to about 2.0% (w/w).

Lower concentrations of zinc and copper salicylate (between about 0.05-0.3% (w/w) of each salt) are used to prevent unacceptable taste, insolubility and mucosal irritation. Zinc salicylate at higher concentrations either alone (4.0%) or in combination with zinc stearate and zinc gluconate (0.5%) has been shown to have anti-inflammatory properties (see U.S. Pat. No. 5,985,918). Use of higher concentrations of zinc salicylate may cause burning and ulceration on the oral mucosa. Lower concentrations of zinc gel (zinc gluconate and zinc lactate panthenol and a hydrogel) alone may not be sufficient for reducing the irritation and inflammation on the oral cavity due to ketorolac. Therefore, in a specific embodiment, a range of between about 0.05-0.3% zinc salicylate is used in the oral composition.

The anti-irritant zinc gel may also be used along with zinc salicylate in an oral composition (zinc salicylate gel complex). This may exhibit enhanced anti-irritant and anti-inflammatory properties and further reduce the ulcerative properties of irritating agents. In nonlimiting embodiments, oral composition may further comprise chlorophyllin and/or spearmint oil which are also known to have anti-inflammatory, anti-irritant and wound healing properties as the coloring agent and flavoring agent respectively.

The taste enhancing agent of the present invention may be one or more of a sweetener of flavorant. Any natural or synthetic flavorant or food additive, such as those described in Chemicals Used in Food Processing, Pub. No. 1274, National Academy of Sciences, pages 63-258 (the entire disclosure of which is herein incorporated by reference) can be used. Suitable flavorants include wintergreen oil (methyl salicylate), oil of peppermint, sassafras, anise, spearmint, menthol, fruit flavors, vanilla, cinnamon, spices, flavor oils and oleoresins, as known in the art. The amount of flavorant employed is normally a matter of preference, subject to such factors as flavor type, individual flavor, and strength desired. Preferably, the oral composition comprises from about 0 to about 0.3% flavorant.

Sweeteners useful in the present invention include sucrose, fructose, and levulose, Splenda, aspartame, xylitol and saccharine. Preferably, the oral composition comprises sweeteners in an amount from about 0.1 to about 0.3% (w/w).

In another embodiment of the present invention, the oral compositions further comprise an agent that would otherwise cause mucosal irritation (“irritating agent”), including but not limited to ketorolac, peroxide, surfactant, alcohol (more than 20 percent w/v), microabrasives (e.g., as used in whitening toothpastes), or an essential oil such as spearmint, wintergreen, peppermint, or lavender oil. In one embodiment, the irritating agent is ketorolac or a salt thereof. Ketorolac and its salts are disclosed in U.S. Pat. Nos. 5,464,609, 5,785,951, and 5,646,174, the disclosure of which is incorporated herein in their entirety by reference.

The effective amount of irritating agent used is one that is an anti-inflammatory amount. As used herein, “anti-inflammatory” refers to a substance or treatment that reduces inflammation. Inflammation is the first response of the immune system to infection or irritation, and is characterized by redness, heat, swelling, pain and dysfunction of the organs involved. In a specific embodiment, the anti-inflammatory amount of the irritating agent is between about 0.1% to about 0.5% (w/w).

In another embodiment of the present invention, the salicylate salts may be formulated along with curcumin and its derivatives, preferably in purified form. Curcumin and curcuminoids are present in turmeric which is a plant product and is used in food preservation and coloring; these compounds are generally recognized for their antioxidant properties. It has also been reported to have anti-inflammatory and antimicrobial properties. (Safety and Anti-Inflammatory Activity of Curcumin: A Component of Tumeric (Curcuma longa), N Chainani-Wu, Journal of Alternative and Complementary Medicine, 2003—liebertonline.com. and Turmeric and the Healing Curcuminoids (Book), M Majeed, V Badmaev, F Murray; 1999, McGraw-Hill, U.S. Pat. No. 5,861,415).

The composition of the present invention may be formulated into solutions, suspension, gels, or other liquid or nonliquid forms. Thus, the compositions may further contain nonionic, cationic surfactants such as Pluronic, Tween, quaternary ammonium compounds, thickening agents and gelling agents, humectants, preservatives and appropriate pharmaceutically acceptable carriers.

Thickening agents in amounts ranging from about 0.05% to about 5% may be used. Examples include, but are not limited to hydroxyethyl cellulose and water, soluble salts of cellulose ethers, including sodium carboxymethyl cellulose and sodium carboxy methylhydroxyethyl cellulose; or natural gums including gum karaya, gum Arabic, and gum tragacanth. Also colloidal magnesium aluminum silicate or finely divided silica maybe be used.

A hydrogel, as used herein, may comprise hydroxypropylmethyl cellulose, cationic hydroxyethyl cellulose (U-care polymers), ethyl cellulose, hydroxypropyl cellulose, hydroxymethyl cellulose, carboxy methyl cellulose, polyethylene oxide (polyox resins), or chitosan pyrrolidone carboxylate (Kytomer PC). These hydrogels preferably do not adversely bind to any added antimicrobial agent, therefore leaving the optionally added antimicrobial agent free for rapid and long-term activity. In addition, it has been discovered that alcohol used to form the hydroalcoholic gel is not trapped in the hydroalcoholic gel composition and is therefore available for rapid and long-term action. The hydrogel may be present in a concentration between 0.1-1.0%, and preferably is a cationic hydroxyethyl cellulose (U-care polymers) in a concentration between 0.05-0.5%, most preferably 0.2%.

Humectants in amounts ranging from about 2% to about 20% may be used. Examples of humectants include but are not limited to glycerin, sorbitol, and other edible polyhydric alcohol or mixtures thereof.

Surfactants may also be used in amounts ranging from about 0% to about 2.0%. Examples of surfactants include but are not limited to pluronic, polyethylene oxide condensates of alkyl phenols, the polyoxyethylene derivatives of fatty acid partial esters of sorbitol anhydride and available in the market under the trade name “Tween”, quaternary ammonium compounds having one long alkyl chain containing from about 8 to about 18 carbon atoms such as lauryl trimethylammonium chloride, cetyl pyridinium chloride, cetyl trimethylammonium bromide, di-isobutylphenoxyethyl-dimethylbenzylammonium chloride, coconut alkyltrimethyl ammonium nitrite, cetyl pyridinium fluoride and the like.

Preservatives in the amount ranging from about 0.05% to about 0.3% may be used. Examples include but are not limited to chlorhexidine, triclosan, sorbic acid potassium sorbate.

Ethanol in amounts ranging from about 0% to about 15% may be used, and the balance with water and coloring agents (e.g., chlorophyllin) which are compatible with the salts.

### Methods of Use

The compositions of the present invention are used in the treatment and prevention of ulcer in the oral mucosa and stomach, and prevention of oral mucosal ulceration. The invention provides the advantage of using the composition along with irritating agents, such as ketorolac, for the treatment of oral disease without causing ulcer and irritation.

The compositions of the present invention may be used in patients who currently require treatment for mucosal irritations or ulcerations, for example for treatment of periodontitis. In another embodiment, the compositions may be used in patients for the prevention of the development of future mucosal irritations. It is known in the art that chronic periodontitis is a significant contributor to circulating TNF. In particular, the present invention has unexpectedly found that by lowering GCF-IL-1 beta in diabetic patients with chronic periodontitis, either by operation or with an adjunctive rinse or both, circulating TNF levels are lowered and insulin resistance is significantly reduced, thus a more desirable level of glycemic control is achieved.

The composition may be formulated for use in mouthwashes, mouth sprays, toothpastes, chewing gums, hard candies, lozenges, torches, lollipops, or other solutions. Furthermore, the compositions may be used on devices including but not limited to pacifiers, dental floss, toothpicks, toothbrushes, dental go between brushes, or chewable pet toys. The compositions may also be applied to dental solutions or devices, including for example implants. In other embodiments of the invention, the compositions may be applied to central venous catheters, soft tissue patches, peritoneal dialysis catheters, and knee and hip implants.

### Definitions

As used herein, a “therapeutically effective amount” means the amount of a compound that, when administered to a mammal for treating a state, disorder or condition is sufficient to effect such treatment. The “therapeutically effective amount” will vary depending on the compound, the disease and its severity and the age, weight, physical condition and responsiveness of the mammal to be treated. According to the present invention, in one embodiment, a therapeutically effective amount of an irritation-inhibiting amount of compound is an amount effective to treat or prevent mucosal ulcerations or other mucosal irritations. Other uses include, but are not limited to, the treatment of periodontal disease. The effective amount of the drug for pharmacological action, and therefore the composition strength, depends on the disease itself.

As used herein, the term “pharmaceutically acceptable” refers to biologically or pharmacologically compatible for in vivo use, and preferably means approved by a regulatory agency of the Federal or a state government or listed in the U.S. Pharmacopeia or other generally recognized pharmacopeia for use in animals, and more particularly in humans.

As used herein, the term “treat” and its derivatives are used herein to mean to relieve or prevent mucosal irritations. The term “treat” may mean to relieve or alleviate the intensity and/or duration of a manifestation of disease experienced by a subject in response to a given stimulus (e.g., pressure, tissue injury, cold temperature, etc.). For example, in relation to periodontal disease, the term “treat” may mean to treat oral ulcerations. Within the meaning of the present invention, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (.+−.20%) are more applicable. Thus, 6 hours can be, e.g., 4.8 hours, 5.5 hours, 6.5 hours, 7.2 hours, as well as the usual 6 hours.

The present invention will be better understood by reference to the following Examples, which are provided as exemplary of the invention, and not by way of limitation.

### EXAMPLES

### Example 1

### Toxicity Testing of Ketorolac

The following toxicity test from the NAMSA (North American Science Associates, Ohio) is carried out to determine what concentrations of ketorolac would show toxicity in this test (positive control). The following test groups are incorporated to determine the level of toxicity of ketorolac including microhistopathology.

The test is carried out to determine the level of intradermal toxicity. The samples are injected intradermally on the back of rabbits. 7 days later, the skin reactions are observed. Edema and erythmia are then scored. Skin samples will also be removed for microhistopathology.

The Intracutaneous Reactivity Test is a measure of the irritant potential of a material. An instillation of 0.2 ml of either an extract from a material or a direct infusion of a material is placed into the dermal tissue below the epidermis, but above the muscle and fat. The instillation site is then watched and evaluated over a 72-hour period for any local changes caused by a reaction to the instilled material.

In this case, a Ketorolac solution will be instilled both with and without an anti-irritant and the site will be scored for local reaction. Further, some of the sites will be surgically excised, fixed and scored microhistopathologically for any changes that are not evident on the surface. Changes can occur at the microscopic level that may not be presented in the local effect visible on the dermal surface and these changes may indicate irritation increase or amelioration.

By combining the macro and micro effects, this test is a valuable screening test for determining the irritation potential of a given material or combination of materials.

### Test 1: Toxicity of Ketorolac

1) Ketorolac alone 0.2%+microhistopathology
2) Ketorolac alone 0.4%+microhistopathology
3) Ketorolac alone 0.6%+microhistopathology
NOTE 1: The concentration which gives a significant positive result will be used for testing against various anti-inflammatory compounds and combinations.
NOTE 2: If a significant irritation effect is not produced higher concentrations of Ketorolac will be tested.
   Test 2: Toxicity of Anti Inflammatory Agents with and without Ketorolac
   Compounds C, Zinc Salicylate (ZnSal), Zinc Gluconate (ZnG) and Zinc Lactate (ZnL). The % of each ingredient shown singly will be the % used throughout Test 2 for each ingredient.

### A. First Set for Study

### 4) C (0.2%)

### 5) ZnG (0.2%)

### 6) ZnL (0.4%)

### 7) ZnSal (0.2%)

### 8) ZnG+ZnL

### 9) ZnG+ZnSal

### 10) ZnL+ZnSal

### 11) ZnG+ZnL+ZnSal

### 12) ZnG+C

### 13) ZnL+C

### 14) ZnSal+C

### 15) ZnG+ZnSal+C

### 16) ZnL+ZnSal+C

### 17) ZnG+ZnL+C

### 18) ZnG+ZnL+ZnSal+C

### 19) Zytrel™ [ZnG (0.2%)+ZnL (0.4%)+Panthenol (0.5%)+Methocel (0.2%)]

### 20) Zytrel™+ZnSal

### 21) Zytrel™+C

### 22) Zytrel™+ZnSal+C

### B Second Set for Study the % is as Shown Previously

If any of the above group shows toxicity, those will be excluded for studies with ketorolac as per the following:

### 23) C (0.2%)+Ketorolac

### 24) ZnG (0.2%)+Ketorolac

### 25) ZnL (0.4%)+Ketorolac

### 26) ZnSal (0.2%)+Ketorolac

### 27) ZnG+ZnL+Ketorolac

### 28) ZnG+ZnSal+Ketorolac

### 29) ZnL+ZnSal+Ketorolac

### 30) ZnG+ZnL+ZnSal+Ketorolac

### 31) ZnG+C+Ketorolac

### 32) ZnL+C+Ketorolac

### 33) ZnSal+C+Ketorolac

### 34) ZnG+ZnSal+C+Ketorolac

### 35) ZnL+ZnSal+C+Ketorolac

### 36) ZnG+ZnL+C+Ketorolac

### 37) ZnG+ZnL+ZnSal+C+Ketorolac

### 38) Zytrel™ [ZnG+ZnL+Panthenol (0.5%)+Methocel (0.2%)]+Ketorolac

### 39) Zytrel™+ZnSal+Ketorolac

### 40) Zytrel™+C+Ketorolac

### 41) Zytrel™+ZnSal+C+Ketorolac

### C. Third Set for Study

If the above groups were found to be non-toxic, the following groups will be tested:
42) Mouth wash Base (Glycerin. Panthenol, Pluronic, Sorbic Acid, Potassium Sorbate Saccharin, Spearmint)

### 43) Mouthwash Base+Zytrel™+ZnSal+C

44) Mouthwash Base+Zytrel™+ZnSal+C+Ketorolac* *The Ketorolac % chosen from Test 1

### D. Second Set for Study: 5 Groups

If the combination groups in the first set show toxicity, the experiment will be repeated with 50% less concentration of 5 important groups.

### Example 2

### An Anti-Irritant Anti-Inflammatory Mouth Wash Containing Zinc Salicylate and Zinc-Hydrogel and Ketorolac

Oral compositions for the safe use of ketorolac were developed to significantly reduce the irritant and ulcerative effect of ketorolac. Formulations containing zinc salicylate with the anti-irritant, anti-inflammatory zinc-hydrogel (lower concentrations of zinc gluconate and zinc lactate, along with panthenol and hydrogel) were prepared. Chlorophyllin was used as the coloring agent and spearmint oil was used as the flavoring agent.

The following 2 prototype formulas were prepared:

Composition (%)

| | Mouth wash | Mouth wash |
|---|---|---|
| Ingredients | Z-K #4 | Z-K #5 |
| Water | 80 | 92 |
| Zinc gluconate | 0.3 | 0.3 |
| Zinc lactate | 0.3 | 0.3 |
| Zinc salicylate | 0.2 | 0.2 |
| Methocel (K4MS) | 0.2 | 0.2 |
| Panthenol (50 W) | 1.0 | 1.0 |
| Glycerin | 5.0 | 5.0 |
| Sodium Saccharin | 0.08 | 0.08 |
| Pluronic F127 | 0.3 | 0.3 |
| Sorbic acid | 0.1 | 0.1 |
| Potassium Sorbate | 0.1 | 0.1 |
| Ethanol (95%) | 12.0 | — |
| Ketorolac Tromethamine | 0.2 | 0.2 |
| Spearmint oil | 0.01 | 0.01 |
| Chlorophyllin* | 0.004 | 0.004 |

*= 0.4% of 1.0% chlorophyllin was used

### Example 3

### Compositions of Mouthwash Containing Ketorolac and Zinc-Hydro Gel

The following oral compositions were developed to significantly reduce the irritant and ulcerative effect of ketorolac.

| | Mouth wash | Mouth wash |
|---|---|---|
| Ingredients | Z-K #6 | Z-K #7 |
| Water | 80.4 | 92.4 |
| Zinc gluconate | 0.1 | 0.1 |
| Zinc lactate | 0.2 | 0.2 |
| Zinc salicylate | 0.1 | 0.1 |
| Methocel (K4MS) | 0.2 | 0.2 |
| Panthenol (50 W) | 1.0 | 1.0 |
| Glycerin | 5.0 | 5.0 |
| Sodium Saccharin | 0.08 | 0.08 |
| Pluronic F127 | 0.3 | 0.3 |
| Sorbic acid | 0.1 | 0.1 |
| Potassium Sorbate | 0.1 | 0.1 |
| Ethanol (95%) | 12.0 | — |
| Ketorolac Tromethamine | 0.2 | 0.2 |
| Spearmint oil | 0.01 | 0.01 |
| Chlorophyllin* | 0.004 | 0.004 |

### Example 4

### General Composition of Mouthwash Containing Ketorolac and Zinc Hydrogel

The following is the general composition for a mouthwash containing ketorolac and the zinc hydrogel.

| | Range (%) |
|---|---|
| Ingredients | Z-K Mouth wash |
| Water | 70-90 |
| Zinc gluconate | 0.1-3 |
| Zinc lactate | 0.1-5 |
| Zinc salicylate | 0.1-3 |
| Methocel (K4MS) | 0.1-5 |
| Panthenol (50 W) | 0.5-1.0 |
| Glycerin | 2.0-10.0 |
| Sodium Saccharin | 0.05-0.1 |
| Pluronic F127 | 0.2-0.5 |
| Sorbic acid | 0.05-0.2 |
| Potassium Sorbate | 0.05-0.2 |
| Ethanol (95%) | 0-20.0 |
| Ketorolac Tromethamine | 0.1-0.5 |
| Spearmint oil | 0-0.05 |
| Chlorophyllin* | 0-0.008 |

Preparation Method: In a beaker containing the water add the Copper Salicylate; the beaker is placed in a water bath on a magnetic stirrer (40-50° C. temperature). When the ingredient is completely dissolved, add zinc salicylate and methocel. After these are completely dissolved, add glycerin and Pluronic F 127. Mix well. When the Pluronic is dissolved, add sorbic acid and potassium sorbate, and mix well until dissolved. Turn off the heat, add Ketorolac to the ZCS-K formula, and mix well until dissolved. Add spearmint and chlorophyllin, mix.

### Example 5

### Ketorolac and Zinc Salicylate Gel Complex Containing Toothpaste (Z-K Toothpaste)

The following oral compositions containing Zinc hydrogel or tetrahydro-curcuminoid and Zinc gel were prepared.

| Ingredients | % (w/w) |
|---|---|
| Water | qs (100%) |
| Zinc gluconate | 0.3 |
| Zinc lactate | 0.3 |
| Panthenol 50 W | 1.0 |
| Methocel (K4MS) | 0.4 |
| Glycerin | 10.0 |
| Sorbitol (70% in water) | 20.0 |
| Sodium saccharin | 0.2 |
| Magnesium aluminum silicate (veegum regular) | 0.4 |
| Zinc salicylate | 0.2 |
| Zinc stearate | 0.5 |
| Zinc oxide | 0.2 |
| Titanium dioxide | 0.5 |
| Calcium carbonate | 10 |
| Polyoxyethelene sorbitan (20) | 1.5 |
| Ketorolac tromethamine | 0.2 |
| Spearmint oil | 0.1 |
| Chorophyllin | 0.01 |

### Example 6

### Ketorolac, Zinc Salicylate—Zinc-Hydrogel and Curcumin Containing Toothpaste (ZC-K Toothpaste)

The following oral compositions containing Zinc hydrogel or tetrahydro-curcuminoid and Zinc gel were prepared.

| Ingredients | % (w/w) |
|---|---|
| Water | qs (100%) |
| Zinc gluconate | 0.2 |
| Zinc lactate | 0.1 |
| Panthenol 50 W | 1.0 |
| Methocel (K4MS) | 0.2 |
| Glycerin | 10.0 |
| Cocoamido Propyl betaine | 1.5 |
| Sorbitol (70% in water) | 20.0 |
| Sodium saccharin | 0.2 |
| Magnesium aluminum silicate (veegum regular) | 0.4 |
| Zinc salicylate | 0.2 |
| Zinc stearate | 0.5 |
| Zinc oxide | 0.2 |
| Tetrahydrocurcuminoid | 0.5 |
| Titanium dioxide | 0.5 |
| Calcium carbonate | 10 |
| Polyoxyethelene sorbitan (20) | 1.5 |
| Ketorolac tromethamine | 0.2 |
| Spearmint oil | 0.1 |
| Chlorophyllin | 0.01 |

Conclusion: Zinc-hydrogel composition containing soluble zinc salts with zinc salicylate, zinc gluconate and zinc lactate in low concentrations with and without insoluble zinc salts such as zinc stearate and zinc oxide along with panthenol and a hydrogel can be used in oral compositions containing ketorolac to prevent mucosal irritation and ulceration resulting from ketorolac. Curcuminoid compounds which are anti-inflammatory agents can also used along with zinc-hydrogel to potentiate the anti-inflammatory activity. The oral preparation can be mouth rinse and tooth paste.

### Example 7

### Anti-Bacterial Activity of Copper Salicylate and Zinc Salicylate

A culture of Streptococcus mutants (causative bacteria for dental plaque) was grown in brain/heart infusion (BHI). Tubes containing 5 ml of BHI were inoculated with 0.1 ml of 10⁵ CFU bacteria/ml and incubated anaerobically at 37° C. After 24 hours the turbidity was measured; the tubes were subcultured on TSA and incubated for 24 hours and colony counts were measured.

TABLE 1

| | Concentration (ug/ml) | | | | |
|---|---|---|---|---|---|
| Growth | 200 | 100 | 50 | 25 | 0 |
| Copper salicylate | | | | | |
| Visual turbidity | − | ± | + | + | + |
| CFU/ml | 0 | 88 | >10⁶ | >10⁸ | >10⁸ |
| Zinc salicylate | | | | | |
| Visual turbidity | − | − | + | + | + |
| CFU/ml | 0 | 10 | >10⁶ | >10⁸ | >10⁸ |

Conclusion: Both Copper and Zinc Salicylate exhibit anti-Streptococcus activity.

### Example 8

### An Anti-Irritant Anti-Inflammatory Mouth Wash Containing Copper Salicylate

In a preliminary experiment, commercial mouth wash (Generic Brand Shop Rite) was purchased and 0.1% copper salicylate was added.

Evaluation in Volunteers

4 volunteers who complained of gum inflammation and soreness were selected for this evaluation; this study was done during a period of 12 months, each volunteer had the problem at different times. The volunteers used were asked to use the generic brand mouth wash without the copper salicylate twice daily for 2 days with no effect. The volunteers were then asked to use the mouthwash with copper salicylate twice daily; they noticed less pain and inflammation in two days. They were asked to continue the treatment for 7 days. The results are given below:

TABLE 2
Effect of Copper Salicylate Mouth wash on the inflammation of gums

| Volunteer | % reduction of Symptoms | Days of treatment |
|---|---|---|
| Volunteer 1 | 80 | 7 |
| Volunteer 2 | 50 | 7 |
| Volunteer 3 | 100 | 5 |
| Volunteer 4 | 75 | 7 |

Conclusion: Mouth wash containing copper salicylate appear to significantly reduce gum inflammation.

### Example 9

### An Anti-Irritant Anti-Inflammatory Mouth Wash Containing Copper Salicylate and Zinc Salicylate

In a preliminary experiment, Shop Rite Brand mouth wash was purchased and 0.1% Copper Salicylate and 0.1% Zinc salicylate was added and allowed to stay for one week to determine the stability.

Evaluation in Volunteers:

5 volunteers participated in this experiment. None of the participants had previously seen a dentist. Each volunteer complained of gum inflammation and soreness. Each Volunteer had the problem at different times. These volunteers used the control mouth wash twice daily for 5 days with no effect. The volunteers were then asked to use the mouthwash with the zinc and copper salicylate. The results are given below:

TABLE 3
Effect of Zinc and Copper Salicylate Mouth wash on the inflammation of
gums

| Volunteer | % reduction of Symptoms | Days of treatment |
|---|---|---|
| Volunteer 1 | 100 | 3 |
| Volunteer 2* | 80 | 4 |
| Volunteer 3 | 0 | 7 |
| Volunteer 4 | 50 | 7 |
| Volunteer 5 | 80 | 7 |

*This volunteer discontinued the treatment after 4 days.

The volunteers were asked to rank the severity of their gum soreness and inflammation and to assess their symptoms. The reduction in the symptoms were described as self assessment only and percent reduction values were given by each individual.
Conclusion: Mouth wash containing zinc-copper salicylate appears to significantly reduce gum inflammation.

### Example 10

### Preparation of a Mouthwash Containing Copper Salicylate Alone, Combination of Copper Salicylate and Zinc Salicylate for Incorporation of Ketorolac

Oral compositions comprising ketorolac were developed. To significantly reduce the irritant and ulcerative effect of ketorolac, zinc and copper salicylate added to 0.05-0.3% concentration. Spearmint oil was used as the flavoring agent. Spearmint oil exhibit anti-inflammatory, anti-irritant and wound healing properties.

The following formulations were prepared.

TABLE 4
General Formula

| Compounds | Concentration Range (% w/w) |
|---|---|
| Copper Salicylate | 0.05-2.0 |
| Zinc Salicylate | 0-2.0 |
| Zinc gluconate | 0-0.5 |
| Zinc Lactate | 0-2.0 |
| Panthenol | 0-1.0 |
| Hydrogel | 0-4.0 |
| Ketorola | 0-1.0 |
| Water | 50-90 |

The composition further contains thickening agents, surfactants/detergents, preservatives, sweeteners, flavors, buffering agents, and ethanol. Specific formulations include the following:

Mouth wash A: Contains 0.1% Copper salicylate

Mouth wash B: Contains 0.1% Zinc Salicylate

Mouth wash C: Contains 0.1% Copper salicylate+0.1% Zinc salicylate

Mouth wash D: Mouth wash A+0.2% Ketorolac Tromethamine

Mouth wash E: Mouth wash B+0.2% Ketorolac Tromethamine

Mouth wash F: Mouth wash C+0.2% Ketorolac Tromethamine

Mouth wash G: Mouth wash F+Zinc gel

TABLE 5

| | Composition (%) | | | | | |
|---|---|---|---|---|---|---|
| | Mouth wash | | | | | |
| Ingredients | A | B | C | D | E | F |
| Water | 89.1 | 89.1 | 89.0 | 89.3 | 89.3 | 89.2 |
| Cooper | 0.1 | 0 | 0.1 | 0.1 | 0 | 0.1 |
| salicylate | | | | | | |
| Zinc salicylate | 0 | 0.1 | 0.1 | 0 | 0.1 | 0.1 |
| Methocel | 0.2 | 0.2 | 0.2 | 0.2 | 0.2 | 0.2 |
| K4MS | | | | | | |
| Glycerin | 10 | 10 | 10 | 10 | 10 | 10 |
| Sodium | 0.08 | 0.08 | 0.08 | 0.08 | 0.08 | 0.08 |
| Saccharin | | | | | | |
| Pluronic F127 | 0.3 | 0.3 | 0.3 | 0.3 | 0.3 | 0.3 |
| Sorbic acid | 0.1 | 0.1 | 0.1 | 0.1 | 0.1 | 0.1 |
| Potassium | 0.1 | 0.1 | 0.1 | 0.1 | 0.1 | 0.1 |
| Sorbate | | | | | | |
| Ketorolac | 0 | 0 | 0 | 0.2 | 0.2 | 0.2 |
| Tromethamine | | | | | | |
| Spearmint oil | 0.01 | 0.01 | 0.01 | 0.01 | 0.01 | 0.01 |
| Coloring agent | 0.002 | 0.002 | 0.002 | 0.002 | 0.002 | 0.002 |

### Preparation Method:

In a beaker containing the water add Copper salicylate, the beaker is placed in a water bath on a magnetic stirrer (40-50° C. temperature). When the ingredient is completely dissolved, add zinc salicylate and methocel. After these are completely dissolved, add glycerin and pluronic F 127, and mix well. When pluronic is dissolved, add sorbic acid and potassium sorbate. Mix well until dissolved, then turn off the heat and add ketorolac to the ZCS-K formula; mix well until dissolved. Add coloring agent and spearmint oil and mix.

### Example 11

### Mouth Wash Containing Copper Salicylate, Zinc Salicylate, Zinc-Hydrogel and Ketorolac

Some of the ingredients used in the oral care products such as surfactants, preservatives, etc. may cause mucosal irritation. In order to reduce the formulations containing copper salicylate, zinc salicylate, zinc-hydrogel and ketorolac were prepared.

TABLE 6
Mouth wash G

| Ingredients | Composition (%) |
|---|---|
| Water | 92.6 |
| Zinc gluconate | 0.1 |
| Zinc lactate | 0.1 |
| Zinc salicylate | 0.1 |
| Copper salicylate | 0.1 |
| Methocel (K4MS) | 0.2 |
| Panthenol (50 W) | 1.0 |
| Glycerin | 5.0 |
| Sodium Saccharin | 0.08 |
| Pluronic F127 | 0.3 |
| Sorbic acid | 0.1 |
| Potassium Sorbate | 0.1 |
| Ketorolac Tromethamine | 0.2 |
| Spearmint oil | 0.01 |
| Coloring agent | 0.002 |

### Example 12

### Ketorolac Containing Toothpastes

The following toothpaste compositions containing copper salicylate and zinc salicylate was prepared.

TABLE 7
Toothpaste 1

| Ingredients | % (w/w) |
|---|---|
| Water | qs* (100%) |
| Zinc salicylate | 0.1 |
| Copper salicylate | 01 |
| Methocel K4MS | 0.4 |
| Glycerin | 10.0 |
| Sorbitol (70% in water) | 20.0 |
| Sodium saccharin | 0.2 |
| Magnesium aluminum silicate (veegum regular) | 0.4 |
| Zinc oxide | 0.1 |
| Cocoamido Propyl betaine | 1.5 |
| Titanium dioxide | 0.5 |
| Calcium carbonate | 10 |
| Polyoxyethelene sorbitan (20) | 1.5 |
| Ketorolac tromethamine | 0.2 |
| Spearmint oil | 0.1 |
| Coloring agent | 0.01 |

(qs* = Quantum sufficiat, as much as is required to achieve 100%)

The following oral composition containing Ketorolac, Copper salicylate and Zinc salicylate and tetrahydro Curcuminoid was prepared:

TABLE 8
Toothpaste 2

| Ingredients | % (w/w) |
|---|---|
| Water | qs* (100%) |
| Copper Salicylate | 0.2 |
| Methocel (K4MS) | 0.2 |
| Glycerin | 10.0 |
| Cocoamido Propyl Betaine | 1.5 |
| Sorbitol (70% in water) | 20.0 |
| Sodium saccharin | 0.2 |
| Magnesium aluminum silicate (veegum regular) | 0.4 |
| Zinc salicylate | 0.2 |
| Zinc stearate | 0.5 |
| Zinc oxide | 0.2 |
| Tetrahydro Curcuminoid | 0.5 |
| Titanium dioxide | 0.5 |
| Calcium carbonate | 10 |
| Polyoxyethelene sorbitan (20) | 1.5 |
| Ketorolac tromethamine | 0.2 |
| Spearmint oil | 0.1 |
| Coloring agent | 0.01 |

(qs* = Quantum sufficiat, as much as is required to achieve 100%)

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. An oral care formulation comprising an anti-irritant amount of copper salicylate and an effective amount of a taste enhancing agent.

2. The oral care formulation of claim 1, where the anti-irritant amount of copper salicylate is between about 0.05% to about 0.3%

3. The oral care formulation of claim 1, where the taste enhancing agent is selected from the group consisting of a sweetener and a flavorant.

4. The oral care formulation of claim 1, further comprising an anti-inflammatory amount of ketorolac.

5. The oral care formulation of claim 1, further comprising curcurmin.

6. The oral care formulation of claim 1, comprising an agent selected from the group consisting of peroxide, surfactant, microabrasives, more than 20 percent (weight/volume) alcohol, and an essential oil.

7. An oral care formulation comprising an anti-irritant amount of a combination of copper salicylate and zinc salicylate and an effective amount of a taste enhancing agent.

8. The oral care formulation of claim 7, where the amount of copper salicylate is between about 0.05% to about 0.3%.

9. The oral care formulation of claim 7, where the amount of zinc salicylate is between about 0.05% to about 0.3%.

10. The oral care formulation of claim 7, where the taste enhancing agent is selected from the group consisting of a sweetener and a flavor.

11. The oral care formulation of claim 7, further comprising an anti-inflammatory amount of ketorolac.

12. The oral care formulation of claim 7, further comprising cucurmin.

13. The oral care formulation of claim 7 comprising an agent selected from the group consisting of peroxide, surfactant, microabrasives, more than 20 percent (weight/volume) alcohol, and an essential oil.

14. An oral care formulation comprising an anti-irritant amount of one or more water-soluble zinc salt, an anti-inflammatory amount of ketorolac and a an effective amount of a taste enhancing agent.

15. The oral care formulation of claim 14, where the water-soluble zinc salt is selected from the group consisting of zinc lactate, zinc gluconate, and zinc salicylate.

16. The oral care formulation of claim 14, where the amount of zinc salt(s) is between about 0.3% to about 2.0%.

17. The oral care formulation of claim 14, where the taste enhancing agent is selected from the group consisting of a sweetener and a flavor.

18. The oral care formulation of claim 14, further comprising a water insoluble zinc salt.

19. The oral care formulation of claim 14, comprising an agent selected from the group consisting of peroxide, surfactant, microabrasives, more than 20 percent (weight/volume) alcohol, and an essential oil.

20. The oral care formulation of claim 14, further comprising cucurmin.

21. A mouthwash comprising an oral care formulation according to claim 1.

22. A toothpaste comprising an oral care formulation according to claim 1.

23. A chewing gum comprising an oral care formulation according to claim 1.

24. A solution for application to the oral mucosa or tongue comprising an oral care formulation according to claim 1.

25. A device to which has been applied an oral care formulation according to claim 1.

26. The device of claim 25 which is a pacifier.

27. The device of claim 25 which is dental floss.

28. The device of claim 25 which is a pet toy.

29. A method of inhibiting oral mucosal irritation in a subject, comprising treating the oral mucosa of the subject with an effective amount of an oral care formulation according to claim 1.
